(19) United States
(12) Patent Application Publication
(10) Pub. No.: US 2026/0363750 A1
(43) Pub. Date: Nov. 19, 2026

(54) CATHETER ASSEMBLIES AND METHODS OF PERFORMING ELECTROPORATION

(71) Applicant: BARD PERIPHERAL VASCULAR, INC., Franklin Lakes, NJ (US)

(72) Inventor: Samuel Michael Shreve, Scottsdale, AZ (US)

(73) Assignee: BARD PERIPHERAL VASCULAR, INC., Franklin Lakes, NJ (US)

(21) Appl. No.: 19/478,347

(22) PCT Filed: May 9, 2023
(86) PCT No.: PCT/US2023/021521

§ 371 Date: Oct. 24, 2025

(51) Int. Cl. A61B 18/00; A61B 17/00

(52) U.S. Cl. CPC A61B 18/00; A61B 2017/00867; A61B 2018/0016; A61B 2018/00172; A61B 2018/00184; A61B 2018/00214; A61B 2018/00613; A61B 2018/00767

(57) ABSTRACT

A catheter assembly includes an inner catheter assembly and an outer catheter disposed about the inner catheter assembly and being retractable between an extended position and a retracted position. A plurality of self-expanding spokes are affixed to the inner catheter assembly, and a plurality of electrode disks are mounted on the plurality of self-expanding spokes. The plurality of self-expanding spokes have a low-profile position between the outer catheter and the inner catheter assembly when the outer catheter is in the extended position, and the plurality of self-expanding spokes move to an expanded position when the outer catheter is in the retracted position.

### TECHNICAL FIELD

The present disclosure relates to catheter assemblies and methods, and, more particularly, to catheter assemblies and methods for performing electroporation.

### BACKGROUND

Tissue ablation procedures, such as electroporation, are typically conducted by utilizing an electric field to treat diseased cells. To perform an electroporation procedure, a probe and needle assembly, or other similar device, may be inserted into a target area where the diseased cells are located. Once the assembly is positioned, an electric field may be generated at the target area to provide controlled electrical impulses to the diseased cells in order to induce apoptosis or necrosis within the diseased cells. By increasing the number, strength, and duration of the electrical impulses, the electroporation procedure may be adjusted to treat a particular type of diseased cell. Although current electroporation devices may be effective at treating diseased cells, standard electroporation devices must be driven into a target area via an external surface to access diseased cell sites. Accordingly, current electroporation devices are incapable of accessing diseased cells located at particular target areas, such as in intravascular target areas.

### SUMMARY

An object of the present disclosure is to provide a catheter assemblies and methods for performing intravascular electroporation on diseased tissue located within a vascular system.

In one embodiment, a catheter assembly is disclosed. The catheter assembly includes an inner catheter and an outer catheter disposed coaxially about the inner catheter and being retractable between an extended position and a retracted position. A plurality of self-expanding spokes are affixed to the inner catheter, and a plurality of electrode disks are mounted on the plurality of self-expanding spokes. The plurality of self-expanding spokes are in a low-profile position between the outer catheter and the inner catheter when the outer catheter is in the extended position, and the plurality of self-expanding spokes are released to an expanded position when the outer catheter is in the retracted position.

In another embodiment, a catheter assembly is disclosed. The catheter assembly includes an inner catheter having a distal end and a proximal end, and an outer catheter disposed coaxially about the inner catheter and being retractable between an extended position and a retracted position. At least a first set of self-expanding spokes and a second set of self-expanding spokes are affixed to the inner catheter, such that the first set of self-expanding spokes and the second set of self-expanding spokes are in a low-profile positioned between the outer catheter and the inner catheter when the outer catheter is in the extended position and in an expanded position when the outer catheter is in the retracted position. At least a first electrode disk is mounted to the first set of self-expanding spokes and at least a second electrode disk is mounted to the second set of self-expanding spokes. A generator is coupled to the proximal end of the inner catheter and delivers a voltage across the first electrode disk and second electrode disk

In yet another embodiment, a method of performing electroporation is disclosed. The method may involve inserting a catheter having an inner catheter and an outer catheter disposed coaxially about the inner catheter into a target area, and retracting the outer catheter from an extended position to a retracted position such that a plurality of self-expanding spokes affixed to the inner catheter move from a low-profile position between the outer catheter and the inner catheter to an expanded position. With the self-expanding electrodes in the expanded position, the method may further involve delivering a voltage to a plurality of electrode disks mounted on the plurality of self-expanding spokes, such that an electric field is generated between the plurality of electrode disks. The method may then include affecting electroporation within the target area using the electric field generated between the plurality of electrode disks. After electroporation is completed, the method may further involve removing the pulsed voltage from the plurality of electrode disks mounted on the plurality of self-expanding spokes, extending the outer catheter from the retracted position to the extended position, such that the plurality of self-expanding spokes affixed to the inner catheter move from the expanded position to the low-profile position between the outer catheter and the inner catheter, and withdrawing the catheter from the target area.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a perspective view of a catheter assembly including an outer catheter and an inner catheter, according to one or more embodiments shown and described herein;

FIG. 2A depicts a perspective view of the catheter assembly of FIG. 1 with the outer catheter in an extended or partially extended position, according to one or more embodiments shown and described herein;

FIG. 2B depicts a perspective view of the catheter assembly of FIG. 1 with the outer catheter in a retracted or partially retracted position, according to one or more embodiments shown and described herein;

FIG. 3A depicts a perspective view of a catheter assembly with an inner shaft having a movable electrode disk in a first position, according to one or more embodiments shown and described herein;

FIG. 3B depicts a perspective view of the inner shaft of FIG. 3B having a movable electrode disk in a second position, according to one or more embodiments shown and described herein;

FIG. 4 depicts a schematic view of a controller for operating the catheter assembly of FIG. 1, according to one or more embodiments described herein;

FIG. 5 depicts an illustrative flow diagram of a method of performing electroporation, according to one or more embodiments shown and described herein.

FIG. 6A depicts a front side view of the catheter assembly positioned within a target area, according to one or more embodiments shown and described herein; and

FIG. 6B depicts a front side view of the catheter assembly of FIG. 6A having an outer catheter in a retracted position, according to one or more embodiments shown and described herein.

Reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to catheter assemblies and methods of performing electroporation. In some embodiments, a catheter assembly may include an inner catheter and an outer catheter disposed coaxially about the inner catheter and being retractable between an extended position and a retracted position. A plurality of self-expanding spokes may be affixed to the inner catheter, and a plurality of electrode disks may be mounted on the plurality of self-expanding spokes. The plurality of self-expanding spokes may remain in a low-profile position between the outer catheter and the inner catheter when the outer catheter is in the extended position, and the plurality of self-expanding spokes may be released to an expanded position when the outer catheter is in the retracted position.

Embodiments of the present disclosure may be specifically advantageous for performing intravascular electroporation on diseased tissue (e.g., cancerous tissue, lesions, thrombus, etc.) positioned within a vascular system of a patient. By compressing the plurality of self-expanding spokes between the outer catheter and the inner catheter of the catheter assembly, the catheter assembly may be situated intravascularly while ensuring that the plurality of self-expanding spokes do not contact the vessel as the catheter is being positioned. Furthermore, the plurality of electrode disks mounted on the plurality of self-expanding spokes may expand to contact a wall of the vessel once the catheter assembly is positioned and the outer catheter is retracted, which may allow for more uniform treatment of the vessel.

Embodiments of catheter assemblies and methods for performing electroporation will now be described in more detail herein with reference to the drawings, and where like numbers refer to like structures.

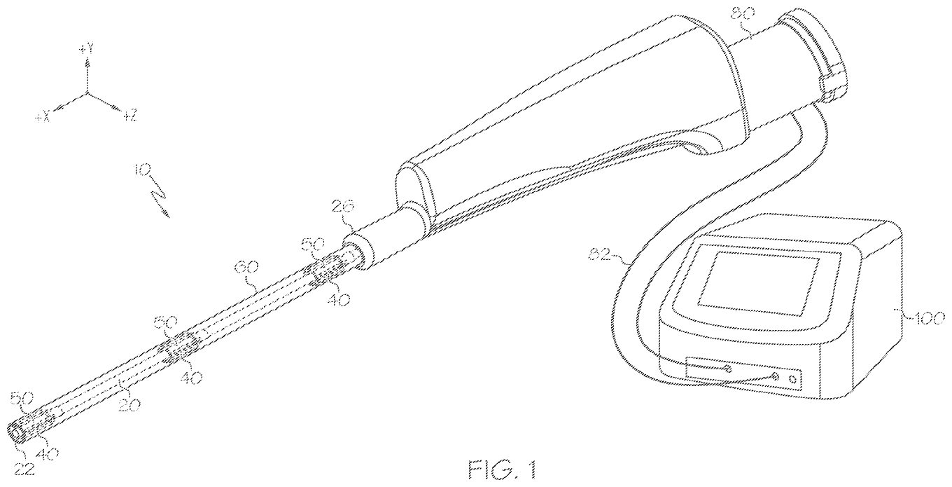

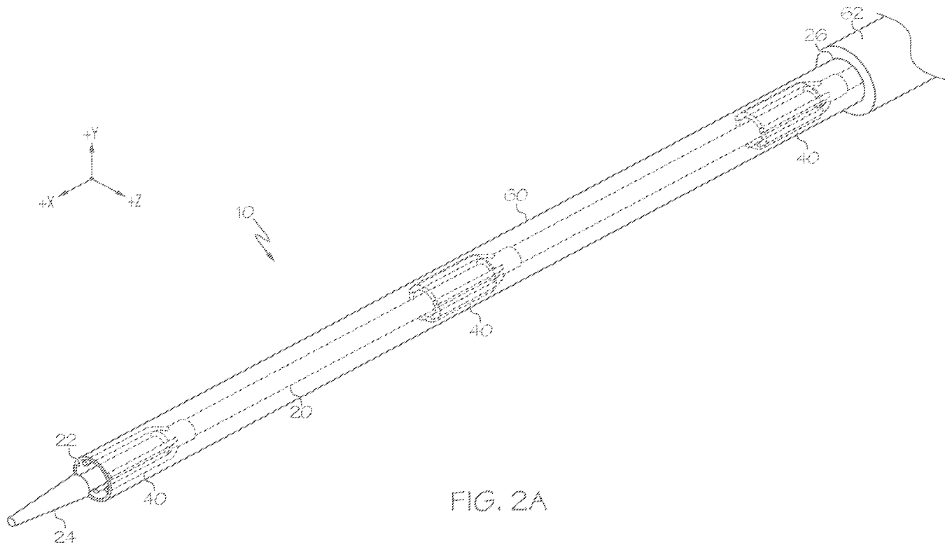

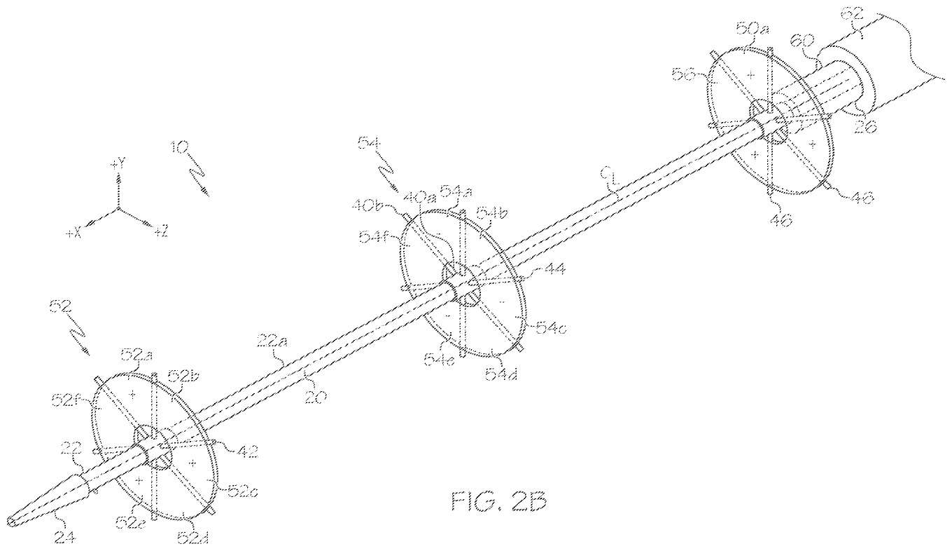

Referring now to FIG. 1, a catheter assembly 10 is disclosed. The catheter assembly 10 may include a catheter, such as an inner catheter 20, having a distal end 22 and a proximal end 26. The catheter assembly 10 may further include a shaft, such as an outer catheter 60, which may be disposed (e.g., coaxially or otherwise) about the inner catheter 20, such that the outer catheter 60 may translate between an extended position and a retracted position, as will be described in additional detail herein with reference to FIGS. 2A and 2B. In the embodiments described herein, the inner catheter 20 may be made of Nylon, polyimide, pebax, PEEK, PTFE, polyurethane, polyethylene, or any other similar materials. Furthermore, in some embodiments, the inner catheter 20 may include a wire having a coiled and/or braided shaft encased within one or more polymer layers.

Referring still to FIG. 1, the inner catheter 20 may further include a plurality of spokes, such as self-expanding spokes 40, fixedly coupled to and, in some embodiments, at least partially penetrating an outer surface 20a of the inner catheter 20. As depicted in FIG. 1, the plurality of self-expanding spokes 40 may be formed of a flexible self-expandable material, such as nitinol wire or any other similar material, such that the plurality of self-expanding spokes 40 may be compressed between the outer catheter 60 and the inner catheter 20 of the catheter assembly 10.

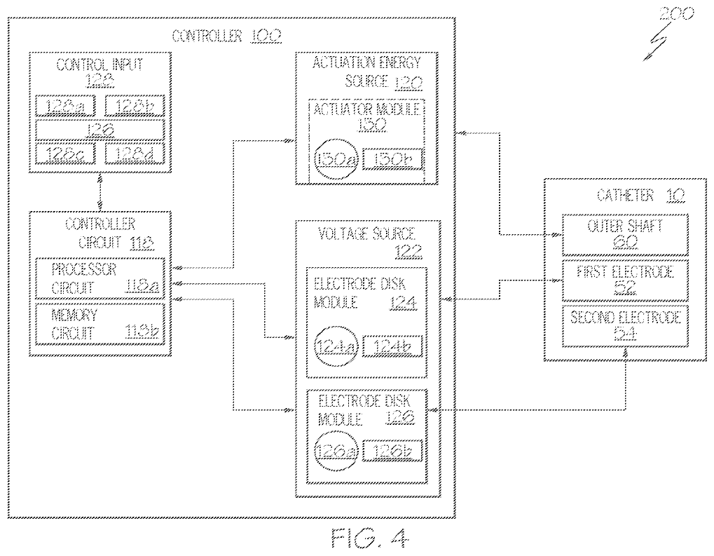

A plurality of electrodes, such as electrode disks 50, may be mounted on the plurality of self-expanding spokes 40, such that the self-expanding spokes 40 expand the electrode disk from a low profile orientation within the outer catheter 60 to an expanded orientation, such as illustrated in FIG. 2B. In these embodiments, the plurality of electrode disks 50 may be deployed when the outer catheter 60 is retracted to the retracted position, as will be described in additional detail herein. With the catheter assembly 10 positioned at a target area such as within a body vessel and the outer catheter 60 retracted, the plurality of electrode disks 50 may expand and be activated to affect electroporation at the target area. In these embodiments, a plurality of electrical leads 82 may extend through the catheter assembly 10 and connect to the plurality of electrode disks 50 such that electricity may be passed to the electrode disks 50, as will be described in additional detail herein with reference to FIG. 4.

Referring still to FIG. 1, the catheter assembly 10 may further include a connector 80, which may be coupled to the proximal end 26 of the catheter assembly 10. In these embodiments, the connector 80 provide a communication harness to communicatively couple the catheter assembly 10 to a controller 100 (illustrated in FIG. 4), which may be used to control operation of the catheter assembly 10. The controller may be otherwise coupled to the catheter assembly 10 to control operation of the catheter assembly 10 without use of a connector 80 (e.g., permanently connected to the catheter assembly 10). For example, the controller 100 may be configured to control actuation of the outer catheter 60 (e.g., between the retracted position and the extended position), activation of the plurality of electrode disks 50, and other similar functions. For example, the catheter assembly 10 may further include a plurality of motors and/or generators, such as electric motors, that may be operated via the controller 100 to control the actuation of the outer catheter 60 between the retracted position and the extended position and/or the electricity supplied to the plurality of electrode disks 50, as will be described in additional detail with reference to FIG. 4.

Turning now to FIGS. 2A and 2B, the catheter assembly 10 is depicted with the outer catheter 60 in an extended position and a retracted position, respectively. As depicted in FIG. 2A, in the extended position, the outer catheter 60 may extend to and cover at least a portion of the distal end 22 of the catheter assembly 10. In these embodiments, the plurality of self-expanding spokes 40 affixed to the outer surface 20a of the inner catheter 20 may remain compressed between the outer catheter 60 and the outer surface 20a of the inner catheter 20.

As further depicted in FIG. 2A, the distal end 22 of the catheter assembly 10 may further include a tip 24. In these embodiments, the tip 24 may be a tapered tip, or any other similarly shaped tip (e.g. atraumatic tip) that may allow for the catheter assembly 10 to navigate a target area (e. g, vasculature). With the plurality of self-expanding spokes 40 compressed between the outer catheter 60 and the inner catheter 20, and the tip 24 concealing the distal end 22 of the inner catheter 20, the catheter assembly 10 may be easily maneuvered and positioned within a target area.

Once the catheter assembly 10 is positioned within the target area, the outer catheter 60 may be retracted from the extended position to the retracted position, as is most clearly depicted in FIG. 2B. In these embodiments, the outer catheter 60 may be translatably coupled to an actuator, or any other similar mechanism capable of linearly actuating the outer catheter 60, such that the outer catheter 60 may be translated in a longitudinal direction (e.g., in the +/−x-direction as depicted in the coordinate axes of FIGS. 2A-2B) between the extended position and the retracted position. For example, in the embodiments described herein, the outer catheter 60 may further include a sliding mechanism including a set of rails or grooves on the inner catheter 20 that guide outer catheter 60 as the outer catheter 60 translates between the extended position and the retracted position. Similarly, the outer catheter 60 may include a screw mechanism including a threaded rod or screw attached to the outer catheter 60. In these embodiments, rotation of the threaded rod or screw may be translated into linear motion, such that the outer catheter 60 may translate between the extended position and the retracted position. In other embodiments, the outer catheter 60 may include a spring loaded mechanism, a hydraulic or pneumatic mechanism, or any other similar mechanism capable of translating the outer catheter 60 between the extended position and the retracted position, as has been described herein.

In the embodiments described above, the catheter assembly 10 may further include a variety of driving mechanisms 62 that may be used to translate the actuator in any of the manners described herein. For example, the catheter assembly 10 may include a motorized driving mechanism 62 (schematically depicted in FIGS. 2A-2B), such as an electric motor, to drive the outer cannula 60 along the set of rails or grooves on the inner catheter 20. In these embodiments, the electric motor may be controlled to adjust the speed and depth that the outer catheter 60 extends and/or retracts. It should be appreciated that an electric motor may be similarly utilized to drive the screw mechanism, such that the electric motor controls rotation of the outer catheter 60 about the inner catheter. Furthermore, in embodiments, in which the outer catheter 60 includes a spring loaded mechanism, the driving mechanism 62 may include a spring loaded driving mechanism. In these embodiments, the spring mechanism may be compressed when the outer cannula 60 is in the retracted position. Accordingly, the spring mechanism may be released to translate the outer cannula 60 to the extended position. Finally, it should also be noted that, in embodiments, the catheter assembly 10 may include a manual driving mechanism, such as a lever or other manual control, that may allow the outer cannula 60 to be translated by hand.

Referring still to FIGS. 2A-2B, as the outer catheter 60 retracts (e.g., in the +x-direction as depicted in the coordinate axis of FIG. 2B), the outer catheter 60 may expose the plurality of self-expanding spokes 40. As the plurality of self-expanding spokes 40 are uncovered, the plurality of self-expanding spokes 40 may deploy from a low-profile position (e.g., in which the plurality of self-expanding spokes and electrode disks are compressed between the outer catheter 60 and the inner catheter 20) to an expanded position, in which the plurality of self-expanding spokes 40 and electrode disks extend radially outwardly from a central axis C_{L} of the inner catheter 20.

In these embodiments, each of the plurality of self-expanding spokes 40 may further include a first end 40a and a second end 40b opposite of the first end 40a. As depicted in FIG. 2B, the first end 40a of each of the plurality of self-expanding spokes 40 may be affixed to the outer surface 20a of the inner catheter 20. In some embodiments, the first end 40a of each of the plurality of self-expanding spokes 40 may be coupled to the outer surface 20a of the inner catheter 20 via welding, brazing, or any other similarly fixed coupling method. In these embodiments, the second end 40b of each of the plurality of self-expanding spokes 40 may be biased radially outwardly, such that, when the outer catheter 60 is retracted, the plurality of self-expanding spokes 40 automatically deploy to the expanded position.

In other embodiments, the first end 40a of each of the plurality of self-expanding spokes 40 may be hingedly coupled to the outer surface 20a of the inner catheter 20. In these embodiments, the catheter assembly 10 may further including a biasing mechanism, such as a spring or other similar mechanism, which biases the second end 40b of each of the plurality of self-expanding spokes 40 in a radially outward direction, such that the plurality of self-expanding spokes 40 automatically deploy to the expanded position when the outer catheter 60 is retracted to the retracted position. In some embodiments, and as noted above, the self-expanding spokes 40 may formed of a shape-memory material that automatically expands upon removal of the compressive outer catheter 60, but in other embodiments where a biasing mechanism is provided and the self-expanding spokes 40 are hingedly coupled to the outer surface 20a, the self-expanding spokes 40 may instead be made of any suitable non-shape-memory material.

Referring still to FIG. 2B, a plurality of electrode disks 50 may be mounted on the plurality of self-expanding spokes 40. In these embodiments, a single electrode disk 50 may be mounted on each set of the plurality of self-expanding spokes 40. The plurality of electrode disks 50 may be formed of a variety of conductive polymers, including polyacetylene (PA), polyaniline (PANI), polypyrrole (PPy), polythiophene (PTH), poly(para-phenylene) (PPP), poly(phenylenevinylene) (PPV), and polyfuran (PF). For example, as depicted in FIG. 2B, the catheter assembly 10 may include a first set of self-expanding spokes 42, a second set of self-expanding spokes 44, and a third set of self-expanding spokes 46. A first electrode disk 52 may be mounted on the first set of self-expanding spokes 42, a second electrode disk 54 may be mounted on the second set of self-expanding spokes 44, and a third electrode disk 56 may be mounted on the third set of self-expanding spokes 46. Although the catheter of FIG. 2B is depicted as having three sets of self-expanding spokes 42, 44, 46 and three electrode disks 52, 54, 56, it should be understood that the catheter assembly 10 may include any number of self-expanding spokes and electrode disks without departing from the scope of the present disclosure. For example, the catheter assembly 10 may include two, four, five, six, or any number of sets of self-expanding spokes and electrode disks as may be necessary to treat diseased tissue positioned at a target area.

Furthermore, although each of the set of self-expanding spokes 42, 44, 46 are depicted as including an electrode disk 52, 54, 56, it should be noted that, in some embodiments, at least one of the sets of self-expanding spokes may not include an electrode disk. For example, the catheter assembly 10 may include a first, second, and third set of self-expanding spokes 40, with the first set of self-expanding spokes 42 including a first electrode disk 52, the second set of self-expanding spokes 44 including a second electrode disk 54, and the third set of self-expanding spokes 46 including no electrode disk. In these embodiments, each of the third set of self-expanding spokes 46 may include or act as an individual electrode for the purpose of affecting electroporation at a target area. It should be appreciated that, in these embodiments, a plurality of electrical leads may extend from the controller to each of the third self-expanding spokes 46 in order to provide electricity to the plurality of self-expanding spokes 46 and allow the plurality of self-expanding spokes to each act as an individual electrode.

Referring still to FIG. 2B, the plurality of electrode disks 50 may be uniformly spaced (e.g., spaced in an equal distance relative one another) in the longitudinal direction (e.g., in the +/−x-direction as depicted in the coordinate axis of FIG. 2B) along the inner catheter 20. By providing uniform spacing between the plurality of electrode disks 50, the catheter assembly 10 may be able to uniformly treat diseased tissue positioned within a target area. However, it should be noted that, in other embodiments, the plurality of electrode disks 50 may be variably spaced in the longitudinal direction along the inner catheter 20. For example, in some embodiments, diseased tissue positioned at a target area may be asymmetrically sized and/or positioned within the target area, such that electroporation may be more efficiently affected by utilizing a plurality of electrode disks 50 having variably spacing along the inner catheter 20.

As further depicted in FIG. 2B, the plurality of electrode disks 50 may be mounted to the plurality of self-expanding spokes 40 such that the plurality of electrode disks 50 extend from the first end 40a to the second end 40b of each of the plurality of self-expanding spokes 40. Accordingly, when the outer catheter 60 is moved to the retracted position, the plurality of electrode disks 50 may similarly move to the expanded position as the plurality of self-expanding spokes 40 deploy. As shown in FIG. 2B, the plurality of electrode disks 50 may expand radially outward from the inner catheter 20, such that the plurality of electrode disks 50 are positioned perpendicularly to the central axis C_{L} of the inner catheter 20 when the plurality of electrode disks 50 are deployed. In these embodiments, the plurality of electrode disks 50 may be mounted to the plurality of self-expanding spokes 40 via brazing, welding, adhesive, or any other similar fixed coupling, such that the plurality of electrode disks 50 deploy when the plurality of self-expanding spokes 40 move to the expanded position.

With the plurality of electrode disks 50 deployed, each of the plurality of electrode disks 50 may be activated in order to affect electroporation at the target area. In these embodiments, each of the plurality of electrode disks 50 may be activated by providing a voltage across the plurality of electrode disks 50, such that an electric field is formed between the plurality of electrode disks 50.

To form the electric field, it should be understood that the voltage may be provided across any pair of the plurality of electrode disks 50 having opposite charges. For example, as depicted in FIG. 2B, the second electrode disk 54 may be a negatively charged electrode disk, while the first electrode disk 52 and the third electrode disk 56 may be positively charged electrode disks. In these embodiments, each of the electrode disks 52, 54, 56 may be coupled to a separate generator that may be configured to provide voltage to each of the electrode disks 52, 54, 56, as will be described in additional detail herein with reference to FIG. 4.

By providing the voltage across oppositely charged adjacent electrode disks, 52, 54, 56, a short and strong electric field may be generated between the first electrode disk 52 and the second electrode disk 54, and between the second electrode disk 54 and the third electrode disk 56, respectively. As the electric field is generated, diseased tissue positioned between the plurality of electrode disks 50 may undergo electroporation treatment (e.g, irreversible electroporation). In the embodiments described herein, the plurality of electrode disks 50 may be constructed of nitinol, conductive polymer, or any other conductive material.

Referring still to FIG. 2B, in some embodiments, only a number of the plurality of electrode disks 50 (e.g., not all of the plurality of electrode disks 50) may be activated to affect electroporation within the target area. Thus, it should be understood that the electric field may only be generated between the plurality of electrode disks 50 that have been activated. For example, in some embodiments, the first electrode disk 52 and the second electrode disk 54 may be activated, while the third electrode disk 56 may remain inactive. In these embodiments, an electric field would only be formed in a region between the first electrode disk 52 and the second electrode disk 54. These embodiments may be particularly advantageous in instances in which the diseased tissue being treated at the target area is positioned between a pair of electrode disks, and does not extend across each of the plurality of electrode disks. It should be noted that, in these embodiments, an electric field may be formed between any two of the plurality of electrode disks 50, so long as opposite voltages are applied to each of the plurality of electrode disks 50 being activated.

Referring still to FIG. 2B, in some embodiments, the plurality of electrode disks 50 may further define a plurality of electrode regions 50a. In these embodiments, the plurality of electrode regions 50a may be defined between each of the plurality of self-expanding spokes 40 used to support the plurality of electrode disks 50. For example, as depicted in FIG. 2B, the first set of self-expanding spokes 42 may include six spokes that may be uniformly mounted about the outer surface 20a of the inner catheter 20. The first electrode disk 52 may define a plurality of electrode regions 52a-f, which may be defined between each of the six spokes mounted about the outer surface 20a of the inner catheter 20.

Although the plurality of self-expanding spokes 40 may be depicted as being uniformly positioned about the outer surface 20a of the inner catheter 20, it should be further understood that, in some embodiments, the plurality of self-expanding spokes 40 may be variably spaced about the outer surface 20a of the inner catheter 20. In these embodiments, the variable spacing of the plurality of self-expanding spokes 40 may cause the plurality of electrode regions 50a on each of the plurality of electrode disks 50 to have differing areas. Accordingly, the catheter assembly 10 may be positioned within the target area such that the area of the electrode region positioned adjacent a lesion corresponds to the volume of the lesion being treated.

Referring still to FIG. 2B, in these embodiments, rather than activating each of the plurality of electrode disks 50, a user may activate at least one of the plurality of electrode regions 50a defined by each of the plurality of electrode disks 50. For example, a user may activate electrode regions 52a, 52c, and 52e of the first electrode disk 52, and electrode regions 54a, 54c, and 54e of the second electrode disk 54. In these embodiments, each of the plurality of electrode regions 52a-f may be insulated from each adjacent electrode region 52a-f in order to allow each of the plurality of electrode regions 52a-f to be activated individually. For example, each of the plurality of spokes 40 may be formed of and/or coated with an insulating material that allows for each of the plurality of electrode regions 52a-f to be exposed for electroporation while also being fully electrically insulated from each adjacent electrode region. Furthermore, in these embodiments, each of the plurality of electrode regions 52a-f may be separately wired in order to allow for a voltage to be applied to a specific electrode region.

Accordingly, in these embodiments, an electric field may only be formed between the plurality of electrode regions 50a that have been activated on each of the plurality of electrode disks 50. By activating only a portion of each of the plurality of electrode disks 50, the electric field generated within the target area may be more precisely controlled, which may be particularly advantageous in instances when the diseased tissue being treated includes a non-continuous or eccentric lesion.

Once the plurality of electrode disks 50 and the relevant electrode regions are activated, it should be understood that the strength of the electric field generated between the plurality of electrode disks 50 may be adjusted by adjusting the voltage supplied to each of the plurality of electrode disks 50. For example, the strength of an electric field generated between two parallel electrode disks (e.g., as depicted in FIGS. 2A and 2B) may be a function of the voltage supplied, the number of pulses, and the pulse duration to each of the electrode disks and a distance between the electrode disks. In these embodiments, increasing the voltage difference between the plurality of electrode disks 50 may increase the strength of the electric field generated between the plurality of electrode disks 50, while decreasing the voltage difference between the plurality of electrode disks 50 may decrease the strength of the electric field generated between the plurality of electrode disks 50. Accordingly, the voltage supplied to the plurality of electrode disks 50 may be adjusted in order to control the strength of the electric field generated between the plurality of electrode disks 50, and in turn, electroporation that is affected at the target area.

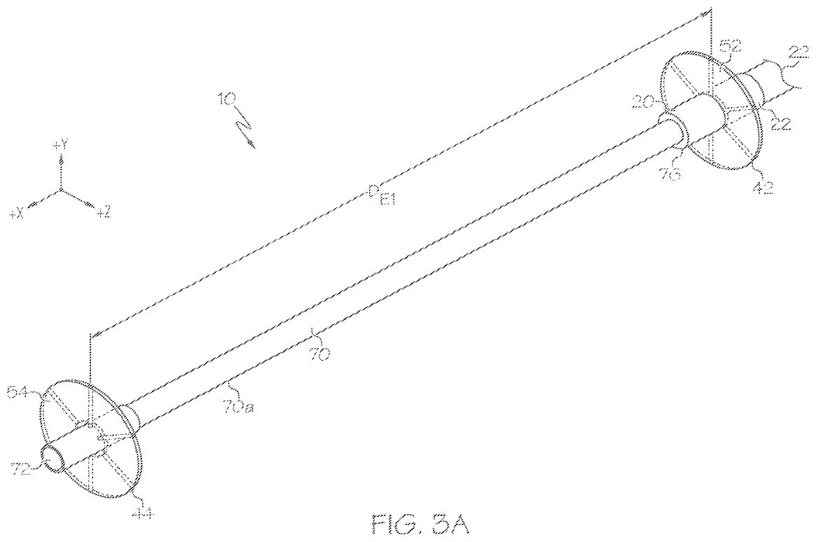

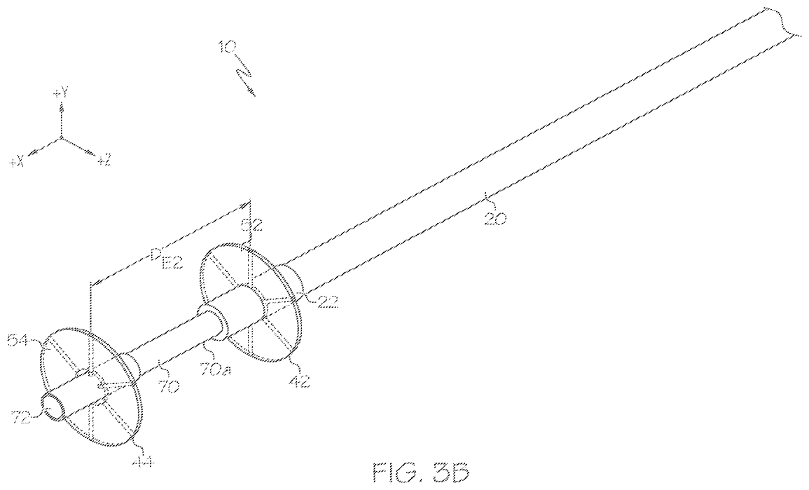

Turning now to FIGS. 3A and 3B, another embodiment of a catheter assembly 10 is disclosed. In these embodiments, the catheter assembly 10 may further include an inner shaft 70 having a distal end 72, a proximal end 76, and an outer surface 70a that extends between the distal end 72 and the proximal end 76 of the inner shaft 70. As depicted in FIGS. 3A and 3B, the inner shaft 70 may be disposed (e.g., coaxially) within the inner catheter 20. The inner shaft 70 and the inner catheter 20 may be defined as an inner catheter assembly In these embodiments, the inner shaft 70 may be translatably disposed within the inner catheter 20, such that the inner shaft 70 may translate in the longitudinal direction (e. g, in the +/−x-direction as depicted in the coordinate axes of FIGS. 3A and 3B) within the inner catheter 20. For example, the inner shaft 70 may be slidably translated within the inner catheter 20, such that inner shaft 70 may translated in the longitudinal direction. In these embodiments, the inner shaft 70 may include separate electrical leads and/or generators which may be configured to control translation of the inner shaft, as will be described in additional detail herein with reference to FIG. 4.

In these embodiments, the catheter assembly 10 may further include a plurality of self-expanding spokes 40 and a plurality of electrode disks 50 mounted to the plurality of expanding spokes, as has been described herein with reference to FIGS. 2A and 2B. For example, the catheter assembly 10 may include at least a first set of self-expanding spokes 42 including a first electrode disk 52 disposed on the distal end 22 of the inner catheter 20 and at least a second set of self-expanding spokes 44 including a second electrode disk 54 disposed on the distal end 72 of the inner shaft 70, as is depicted in FIGS. 3A and 3B. Accordingly, when the outer catheter is moved to the retracted position and the at least first and second sets of self-expanding spokes 42, 44 are deployed to the extended position, the first electrode disk 52 may be deployed at the distal end 22 of the inner catheter 20 while the second electrode disk 54 may be deployed at the distal end 72 of the inner shaft 70. Although the catheter assembly 10 depicted in FIGS. 3A and 3B includes a single set of self-expanding spokes and a single electrode disk on positioned on each of the inner shaft 70 and the inner catheter 20, it should be understood that the inner catheter 20 and the inner shaft 70 may include any number of sets of self-expanding spokes and electrode disks without departing from the scope of the embodiment illustrated in FIGS. 3A and 3B.

Because the inner shaft 70 may be translatably coupled to the inner catheter 20, it should be understood that translation of the inner shaft 70 in the longitudinal direction (e.g., in the +/−x-direction as depicted in the coordinate axes of FIGS. 3A and 3B) may act to adjust a distance between the first electrode disk 52 affixed to the distal end 22 of the inner catheter 20 and the second electrode disk 54 affixed to the distal end 72 of the inner shaft 70.

For example, FIG. 3A depicts the catheter assembly 10 in a first position. In these embodiments, the first electrode disk 52 may be positioned at a first distance D_{E1} in the longitudinal direction from the second electrode disk 54. As the inner shaft 70 is translated in the longitudinal direction, the second electrode disk 54 affixed to the distal end 72 of the inner shaft 70 may translate in the longitudinal direction, such that the catheter assembly 10 is moved to a second position, as is depicted in FIG. 3B. In these embodiments, the first electrode disk 52 may be positioned at a second distance D_{E2} in the longitudinal direction from the second electrode disk 54 when the catheter assembly 10 is moved to the second position.

Referring again to FIGS. 3A and 3B, it should be understood that the inner shaft 70 may be translated in the longitudinal direction in order to adjust the distance between the first electrode disk 52 and the second electrode disk 54 to any desired distance in the longitudinal direction. For example, translating the inner shaft 70 towards the distal end 22 of the inner catheter 20 (e.g., in the −x-direction as depicted in the coordinate axes of FIGS. 3A and 3B) may decrease the distance between the first electrode disk 52 and the second electrode disk 54, while translating the inner shaft 70 away from the distal end 22 of the inner catheter 20 (e.g., in the +x-direction as depicted in the coordinate axes of FIGS. 3A and 3B) may increase the distance between the first electrode disk 52 and the second electrode disk 54. In these embodiments, it should be appreciated that that the distance between the first electrode disk 52 and the second electrode disk 54 may be adjusted to correspond to a particular lesion positioned at the target area, and may further be adjusted during the course of a procedure to treat multiple lesions positioned at the target area.

Furthermore, it should be understood that adjusting the distance between the first electrode disk 52 and the second electrode disk 54 may alter the strength of the electric field generated between the first electrode disk 52 and the second electrode disk 54. As has been described herein with reference to FIGS. 2A and 2B the strength of the electric field generated between the first electrode disk 52 and the second electrode disk 54 may be determinate on the distance between the first electrode disk 52 and the second electrode disk 54. For example, in the embodiments depicted in FIGS. 3A and 3B, decreasing the distance between the first electrode disk 52 and the second electrode disk 54 may increase the strength of the electric field generated between the first electrode disk 52 and the second electrode disk 54, while increasing the distance between the first electrode disk 52 and the second electrode disk 54 may decrease the strength of the electric field generated between the first electrode disk 52 and the second electrode disk 54. Accordingly, in some embodiments, the distance between the first electrode disk 52 and the second electrode disk 54 may be adjusted not only to conform to a particular lesion positioned at the target area, but also to control the strength of the electric field generated between the first electrode disk 52 and the second electrode disk 54.

Turning now to FIG. 4, a schematic view of the controller 100 for operating the catheter assembly 10 is illustrated. In some embodiments, the controller 100 may be configured to actuate the outer catheter 60 between the extended position and the retracted position, and/or control the voltage supplied to the plurality of electrode disks 50. The controller 100 may include a controller circuit 118, an actuation energy source 120, and a voltage source 122, such as a high voltage generator. The controller 100 may further include a control input 128, such as a computer, which may allow a user to operate the controller 100. In these embodiments, the control input 128 may be configured to provide control of the controller 100 via a software program, or may be manually controlled via a user manipulating a user interface thereof.

Referring still to FIG. 4, the control input 128 may include any suitable user interface such as control buttons and/or visual/aural indicators, such as a display and/or speakers, with the user interface (e.g., control buttons) providing user control over various functions of the controller 100, and with the visual/aural indicators providing visual/aural feedback of the status of one or more conditions and/or positions of components of the controller 100. For example, the display and/or speakers may provide an audible tone that indicates to a user that the catheter assembly 10 is positioned within the target area, that the outer catheter 60 has been moved to the extended or retracted position, and/or that the plurality of electrode disks 50 have been activated. The control buttons may include one or more buttons 128a, 128b for actuating the outer catheter 60 between the extended position and retracted position, and one or more buttons and/or knobs for adjusting the voltage supplied to the plurality of electrode disks 50. Furthermore, in embodiments in which the catheter assembly 10 includes the inner catheter 20, the controller 100 may further include one or more buttons for actuating the inner shaft 70 between a first position and a second position, as has been described herein with reference to FIGS. 3A and 3B.

Controller circuit 118 is electrically and communicatively coupled to actuation energy source 120, voltage source 122, and control input 128, such as by one or more wires or circuit traces. Controller circuit 118 may be assembled on an electrical circuit and may include, for example, a processor circuit 118a and a memory circuit 118b.

Processor circuit 118a has one or more programmable microprocessors and associated circuitry, such as an input/output interface, buffers, memory, etc. Memory circuit 118b is communicatively coupled to processor circuit 118a, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), flash memory, etc. Controller circuit 18 may be formed as one or more Application Specific Integrated Circuits (ASIC).

Controller circuit 118 is configured via software and/or firmware residing in memory circuit 118b to execute program instructions to perform functions associated with the actuation of the outer catheter 60, inner shaft 70, and/or voltage supplied to the plurality of electrode disks 50.

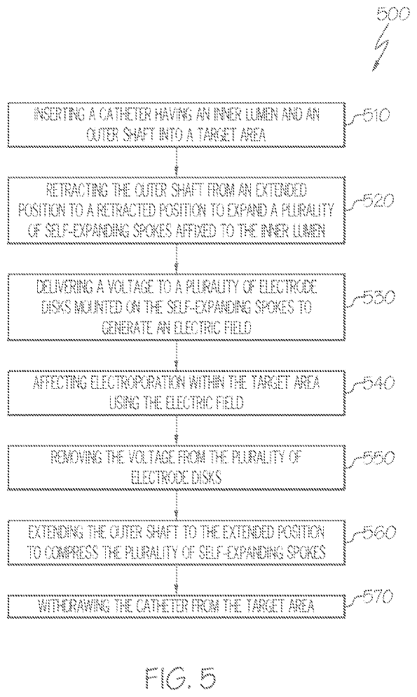

Referring still to FIG. 4, actuation energy source 120 may include, for example, an actuator module 130. In these embodiments, the actuation energy source 120 may include an actuator module 130 for each shaft utilized in the catheter assembly 10. For example, FIG. 5 illustrates a controller 100 for a catheter assembly 10 having only the outer catheter 60. Thus, the actuation energy source 120 includes one actuator module 130. However, in embodiments in which the catheter assembly 10 further include the inner shaft 70, the actuation energy source 120 may include a plurality of actuator modules 130. For example, a controller 100 configured to operate the catheter of FIGS. 3A and 3B may include a first actuator module and a second actuator module, which may be configured to actuate the outer catheter 60 and the inner shaft 70, respectively.

Each of the actuator modules 130 may be electrically and controllably coupled to controller circuit 118. As provided herein, the actuator module 130 may be electrically coupled to the controller circuit 118 by way of electrical wiring or any other suitable electrical connections, such that user inputs on the control input 128 may be relayed to the controller circuit 118 and used to control the power delivered by the actuator module 130 to the outer catheter 60. In these embodiments, the actuator module 130 may include a power supply 130a, such as generator, battery, or similar power supply, to which an electric lead 130b is attached. In embodiments in which multiple actuator modules 130 are present, each actuator module may include a power source and an electric lead.

Referring still to FIG. 4, voltage source 122 may include a plurality of electrode disk modules 124. In these embodiments, the voltage source 122 may include an electrode disk module 124 for each of the plurality of electrode disks 50 positioned on the catheter assembly 10. For example, as depicted in FIG. 5, the voltage source 122 may include a first electrode disk module 124 associated with a first electrode disk 52 and a second electrode disk module 126 associated with a second electrode disk 54. In these embodiments, the first electrode disk module 124 and the second electrode disk module 126 may each include, for example, a power supply 124a, 126a, that provides a voltage to the first electrode disk 52 and the second electrode disk 54, respectively, via an electric lead 124b, 126b. In the embodiments described herein, the voltage source 122 may be a variable voltage source, such that the voltage output by the voltage source 122 may be controllable by a user (e.g., via control input 128 of controller 100, etc.). To effect electroporation, in these embodiments, the voltages output by the voltage source 122 may be up to and including 115 volts (or higher) as may be determined based on the tissue being treated at the target area. Furthermore, the voltage source 122 may provide the voltage output in pulses, such as square waves in the range of 100 microseconds long with a frequency of 10 pulses per second as an example.

It should be understood that each of the first and second electrode disk modules 124, 126 may be electrically and controllably coupled to controller circuit 118. Furthermore, although the first and second electric disk modules 124, 126 and the actuator module 130 are depicted as having separate power supplies 124a, 126a, 130a, respectively, it should be further understood that, in some embodiments, each of the components of the controller 100 may be powered via a single power supply or be connected to a single power source.

Referring still to FIG. 4, in the embodiments described herein, the controller 100 may be electrically and operably connected the proximal end of the catheter assembly 10 via the connector 80. The electric leads 124b, 126b, 130b may extend from the controller 100 through the connector and into the inner catheter 20 of the catheter assembly 10, such that the actuation energy source 120 and the voltage source 122 are able to control the actuation of the outer catheter 60 and the voltage supplied to the plurality of electrode disks 50. For example, once the catheter assembly 10 is positioned within the target area, the actuation energy source 120 may be operated to cause the at least one actuator 132 to move the outer catheter 60 from the extended position to the retracted position, such that the plurality of electrode disks 50 are deployed (as has been described with reference to FIGS. 2A-3B). With the plurality of electrode disks 50 deployed, the voltage source 122 may be operated to supply a voltage to the plurality of electrode disks 50, such that the electric field is formed between the plurality of electrode disks 50. As the plurality of electrode disks 50 are activated, the electric field generated between the plurality of electrode disks 50 may effect electroporation to treat diseased tissue positioned at the target area. Once the tissue positioned within the target area has been treated, the controller circuit 118 may deactivate the voltage source 122 in order to deactivate the plurality of electrode disks 50 and remove the electric field. The controller circuit may then reactivate the actuation energy source 120 such that the outer catheter 60 may be moved from the retracted position to the extended position, at which point the plurality of electrode disks 50 may be compressed between the outer catheter 60 and the inner catheter 20 and the catheter assembly 10 may be safely removed from the target area.

Turning now to FIG. 5, an illustrative flow diagram of a method of performing electroporation 500 is depicted. In these embodiments, the method may first involve inserting a catheter having an inner catheter and an outer catheter disposed coaxially about the inner shaft into a target area, as shown at block 510. In some embodiments, the catheter may be inserted into the target area by inserting the catheter over a guidewire, such that a user is able to more easily position the catheter within the target area. With the catheter positioned at the target area, the method may proceed to block 520, which may involve retracting the outer catheter from an extended position to a retracted position, such that a plurality of self-expanding spokes affixed to the inner catheter flex radially outward from a low-profile position to an expanded position.

In these embodiments, a plurality of electrode disks may be mounted on the plurality of self-expanding spokes, such that, when the plurality of self-expanding spokes move from the low-profile position to the expanded position, the plurality of electrode disks may be deployed at the target site. With the plurality of electrode disks deployed, the method may move to block 530, which may involve delivering a voltage to each of the plurality of electrode disks to generate an electric field between each of the plurality of electrode disks. As has been described herein with reference to FIGS. 2A-2B, the method step of delivering a voltage to each of the plurality of electrode disks may involve delivering avoltage across each pair of the plurality of electrode disks positioned adjacent one another, such that the electric field is generated between adjacent electrodes.

Referring still to FIG. 5, the method may further involve affecting electroporation within the target area using the electric field generated between the plurality of self-expanding spokes, as shown at block 540. In these embodiments, the electroporation affected within the target area may be controlled by adjusting the voltage delivered to each of the plurality of electrode disks, as has been discussed herein with reference to FIG. 4.

Once electroporation has been affected at the target area and diseased tissue has been treated, the method may move to block 550, which may involve removing the voltage from the plurality of electrode disks mounted on the plurality of self-expanding spokes, such that the electric field dissipates. With the electric field dissipated, the method may proceed to block 560, which may include extending the outer catheter from the retracted position to the extended position. As the outer catheter is translated to the extended position, the outer catheter may engage the plurality of self-expanding spokes and force the plurality of self-expanding spokes to move from the expanded position to the low-profile position between the outer catheter and the inner catheter. Once the plurality of self-expanding spokes have been secured in the low-profile position, the catheter may be withdrawn from the target area, as is depicted at block 570.

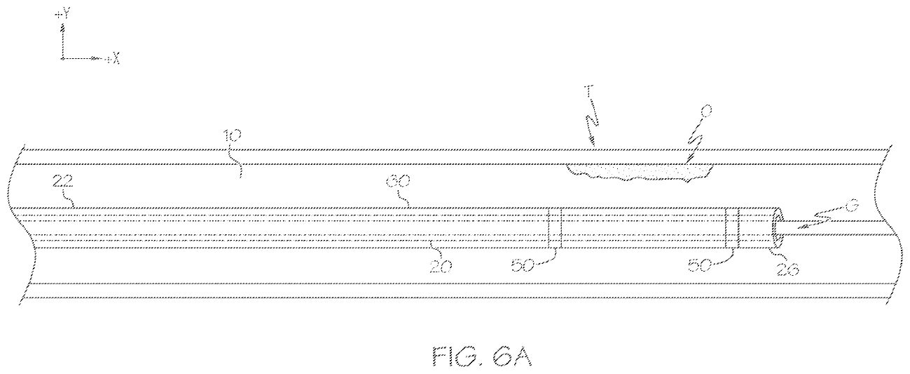

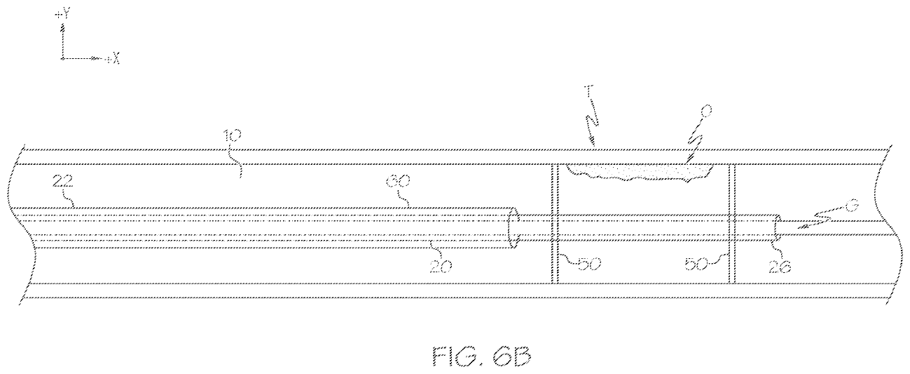

Turning now to FIGS. 6A and 6B, the catheter assembly 10 is depicted positioned within a target site T. As depicted in FIG. 6A, the catheter assembly 10 may be inserted into the target site T over a guidewire G. In these embodiments, the inner catheter 20 may define a lumen, such that the guidewire G may extend through the lumen and guide the catheter assembly 10 into position at the target site T.

Referring still to FIG. 6A, the outer catheter 60 of the catheter assembly 10 is depicted in the extended position. As has been described herein, in these embodiments, the outer catheter 60 may compress the plurality of electrode disks 50 between the outer catheter 60 and the inner catheter 20, such that the plurality of electrodes 50 are in the low-profile orientation. With the outer catheter 60 in the extended position, the catheter assembly 10 may be maneuvered within the target site T until an occlusion O positioned within the target site T is aligned between the plurality of electrodes 50. In these embodiments, a user may utilize fluoroscopy, or any other similar method, to determine when the occlusion O is positioned between the plurality of electrodes 50.

Turning now to FIG. 6B, once the catheter assembly 10 is positioned, the outer catheter 60 may be translated to the retracted position. As has been described herein, when the outer catheter 60 retracts, the plurality of electrodes 50 may become exposed and may automatically deploy to the expanded orientation. As depicted in FIG. 6B, when the plurality of electrodes 50 are deployed, the plurality of electrodes may extend across a diameter of the target site T, such that the occlusion O positioned at the target site T is contained between the plurality of electrodes 50.

With the plurality of electrodes 50 in the expanded orientation, each of the plurality of electrodes 50 may be activated, as has been described herein with reference to FIGS. 2A-2B and 5. As the plurality of electrodes 50 are activated, an electrical field may be generated between the plurality of electrodes 50. Because the occlusion O may be positioned between the plurality of electrodes 50, the electrical field may act upon the occlusion O to affect electroporation. The plurality of electrodes 50 may remain activated until the occlusion O is treated, at which point the plurality of electrodes 50 may be deactivated. For example, during treatment the cells of the occlusion are killed. The remaining material may then deteriorate over time. With the plurality of electrodes 50 deactivated, the outer catheter 60 may be translated to the extended position to compress the plurality of electrodes 50 to the low-profile orientation, and the catheter assembly 10 may be withdrawn from the target site T.

As should be appreciated in view of the foregoing a catheter for affecting electroporation within a target area is disclosed. The catheter includes an inner catheter and an outer catheter disposed coaxially about the inner catheter and being retractable between an extended position and a retracted position. A plurality of self-expanding spokes are affixed to the inner catheter, and a plurality of electrode disks are mounted on the plurality of self-expanding spokes. The plurality of self-expanding spokes are in a low-profile position between the outer catheter and the inner catheter when the outer catheter is in the extended position, and the plurality of self-expanding spokes are released to an expanded position when the outer catheter is in the retracted position. By compressing the plurality of self-expanding spokes between the outer catheter and the inner catheter of the catheter, the catheter may be situated intravascularly while ensuring that the plurality of self-expanding spokes do not contact the target area as the catheter is being positioned. Furthermore, the plurality of electrode disks mounted on the plurality of self-expanding spokes may expand to contact a wall of the target area once the catheter is positioned and the outer catheter is retracted, which may allow for more uniform treatment of the target area.

Embodiments may be further described with references to the following numbered clauses:

Clause 1. A catheter assembly comprising: an inner catheter; an outer catheter disposed coaxially about the inner catheter and being retractable between an extended position and a retracted position; a plurality of self-expanding spokes affixed to the inner catheter; and a plurality of electrode disks mounted on the plurality of self-expanding spokes; wherein the plurality of self-expanding spokes are in a low-profile position between the outer catheter and the inner catheter when the outer catheter is in the extended position and the plurality of self-expanding spokes are released to an expanded position when the outer catheter is in the retracted position.

Clause 2. The catheter assembly of clause 1, wherein the plurality of electrode disks are perpendicular to a central axis of the inner catheter assembly when the plurality of self-expanding spokes are in the expanded position.

Clause 3. The catheter assembly of clause 1 or 2, wherein the plurality of self-expanding spokes comprise a flexible self-expandable material, preferably nitinol wire.

Clause 4. The catheter assembly of clauses 1-3, further comprising a connector coupled to a proximal end of the inner catheter of the catheter.

Clause 5. The catheter assembly of any of clauses 1-4, further comprising one or more generators coupled to the connector, wherein the one or more generators are configured to delivers voltages to the plurality of electrode disks.

Clause 6. The catheter assembly of any of clauses 1-5, wherein the generator is a high voltage generator.

Clause 7. The catheter assembly of any of clauses 1-6, wherein the one or more generators are configured to provide a voltage across a pair of each of the plurality of electrode disks positioned adjacent one another.

Clause 8. The catheter assembly of any of clauses 1-7, wherein the inner catheter further comprises an atraumatic tip.

Clause 9. The catheter assembly of any of clauses 1-8, wherein each of the plurality of electrode disks are spaced an equal distance apart from one another in a longitudinal direction along the inner catheter.

Clause 10. The catheter assembly of any of clauses 1-9, wherein each of the plurality of electrode disks are variably spaced apart from one another in a longitudinal direction along the inner catheter.

Clause 11. The catheter assembly of any of clauses 1-10, wherein the plurality of electrode disks comprises at least a first electrode disk and a second electrode disk, the first electrode disk being movable relative the second electrode disk.

Clause 12. A catheter assembly comprising: an inner catheter assembly comprising at least an inner catheter having a distal end and a proximal end; an outer catheter disposed about the inner catheter assembly and being retractable between an extended position and a retracted position; at least a first set of self-expanding spokes and a second set of self-expanding spokes affixed to the inner catheter, the first set of self-expanding spokes and the second set of self-expanding spokes being in a low-profile position between the outer catheter and the inner catheter assembly when the outer catheter is in the extended position and in an expanded position when the outer catheter is in the retracted position; at least a first electrode disk mounted to the first set of self-expanding spokes and at least a second electrode disk mounted to the second set of self-expanding spokes; and a generator coupled to the proximal end of the inner catheter; wherein the generator is configured to deliver a voltage across the first electrode disk the second electrode disk.

Clause 13. The catheter assembly of clause 12, wherein the first electrode disk and the second electrode disk are perpendicular to a central axis of the inner catheter assembly when the first set of self-expanding spokes and the second set of self-expanding spokes are in the expanded position.

Clause 14. The catheter assembly of clauses 12 or 13, wherein the first set of self-expanding spokes and the second set of self-expanding spokes comprise a flexible self-expandable material, preferably nitinol wire.

Clause 15. A method of performing electroporation comprising: inserting a catheter assembly having an inner catheter and an outer catheter disposed coaxially about the inner catheter into a target area; retracting the outer catheter from an extended position to a retracted position, such that a plurality of self-expanding spokes affixed to the inner catheter move from a low-profile position between the outer catheter and the inner catheter to an expanded position; delivering a voltage to a plurality of electrode disks mounted on the plurality of self-expanding spokes, such that an electric field is generated between the plurality of electrode disks; affecting electroporation within the target area using the electric field generated between the plurality of electrode disks; removing the voltage from the plurality of electrode disks mounted on the plurality of self-expanding spokes; extending the outer catheter from the retracted position to the extended position, such that the plurality of self-expanding spokes affixed to the inner catheter move from the expanded position to the low-profile position between the outer catheter and the inner catheter; and withdrawing the catheter from the target area.

Clause 16. The method of clause 15, wherein the method step of delivering the voltage further comprises delivering an opposite voltage to each of the plurality of electrode disks positioned adjacent one another.

Clause 17. The method of any of clauses 15-16, wherein the plurality of electrode disks mounted on the plurality of self-expanding spokes are perpendicular to a central axis of the catheter when the plurality of self-expanding spokes are in the expanded position.

Clause 18. The method of any of clauses 15-17, wherein each of the plurality of electrode disks are spaced an equal distance apart from one another in a longitudinal direction along the inner catheter.

Clause 19. The method of any of clauses 15-18 wherein each of the plurality of electrode disks are variably spaced apart from one another in a longitudinal direction along the inner catheter.

Clause 20. The method of any of clauses 15-19, wherein the method step of inserting the catheter assembly further comprises inserting the catheter assembly over a guidewire and into the target area.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter assembly comprising:
an inner catheter assembly comprising at least an inner catheter;
an outer catheter disposed about the inner catheter assembly and being retractable between an extended position and a retracted position;
a plurality of self-expanding spokes affixed to the inner catheter; and
a plurality of electrode disks mounted on the plurality of self-expanding spokes;
wherein the plurality of self-expanding spokes have a low-profile position between the outer catheter and the inner catheter assembly when the outer catheter is in the extended position and the plurality of self-expanding spokes move to an expanded position when the outer catheter is in the retracted position.

2. The catheter assembly of claim 1, wherein the plurality of electrode disks are positioned perpendicular to a central axis of the inner catheter assembly when the plurality of self-expanding spokes are in the expanded position.

3. The catheter assembly of claim 1, wherein the plurality of self-expanding spokes comprise a flexible self-expandable material, preferably nitinol wire.

4. The catheter assembly of claim 1, further comprising a connector coupled to a proximal end of the inner catheter of the catheter.

5. The catheter assembly of claim 4, further comprising one or more generators coupled to the connector, wherein the one or more generators are configured to deliver voltages to the plurality of electrode disks.

6. The catheter assembly of claim 5, wherein the generator is a high voltage generator.

7. The catheter of assembly claim 6, wherein the one or more generators are configured to provide a voltage across a pair of each of the plurality of electrode disks positioned adjacent one another.

8. The catheter of assembly claim 1, wherein the inner catheter further comprises an atraumatic tip.

9. The catheter of assembly claim 1, wherein each of the plurality of electrode disks are spaced an equal distance apart from one another in a longitudinal direction along the inner catheter.

10. The catheter of assembly claim 1, wherein each of the plurality of electrode disks are variably spaced apart from one another in a longitudinal direction along the inner catheter.

11. The catheter of assembly claim 1, wherein the plurality of electrode disks comprises at least a first electrode disk and a second electrode disk, the first electrode disk being movable relative the second electrode disk.

12. A catheter assembly comprising:
an inner catheter assembly comprising at least an inner catheter having a distal end and a proximal end;
an outer catheter disposed about the inner catheter assembly and being retractable between an extended position and a retracted position;
at least a first set of self-expanding spokes and a second set of self-expanding spokes affixed to the inner catheter assembly, the first set of self-expanding spokes and the second set of self-expanding spokes being in a low-profile position between the outer catheter and the inner catheter assembly when the outer catheter is in the extended position and in an expanded position when the outer catheter is in the retracted position;
at least a first electrode disk mounted to the first set of self-expanding spokes and at least a second electrode disk mounted to the second set of self-expanding spokes; and
a generator coupled to the proximal end of the inner catheter;
wherein the generator is configured to deliver a voltage across the first electrode disk and a the second electrode disk.

13. The catheter assembly of claim 12, wherein the first electrode disk and the second electrode disk are positioned perpendicular to a central axis of the inner catheter assembly when the first set of self-expanding spokes and the second set of self-expanding spokes are in the expanded position.

14. The catheter assembly of claim 12, wherein the first set of self-expanding spokes and the second set of self-expanding spokes comprise a flexible self-expandable material, preferably nitinol wire.

15. A method of performing electroporation comprising:
inserting a catheter assembly having an inner catheter and an outer catheter disposed coaxially about the inner catheter into a target area;
retracting the outer catheter from an extended position to a retracted position, such that a plurality of self-expanding spokes affixed to the inner catheter move from a low-profile position between the outer catheter and the inner catheter to an expanded position;
delivering a voltage to a plurality of electrode disks mounted on the plurality of self-expanding spokes, such that an electric field is generated between the plurality of electrode disks;
affecting electroporation within the target area using the electric field generated between the plurality of electrode disks;
removing the voltage from the plurality of electrode disks mounted on the plurality of self-expanding spokes;
extending the outer catheter from the retracted position to the extended position, such that the plurality of self-expanding spokes affixed to the inner catheter move from the expanded position to the low-profile position between the outer catheter and the inner catheter; and
withdrawing the catheter assembly from the target area.

16. The method of claim 15, wherein the method step of delivering the voltage further comprises delivering an opposite voltage to each of the plurality of electrode disks positioned adjacent one another.

17. The method of claim 15, wherein the plurality of electrode disks mounted on the plurality of self-expanding spokes are perpendicular to a central axis of the catheter when the plurality of self-expanding spokes are in the expanded position.

18. The method of claim 15, wherein each of the plurality of electrode disks are spaced an equal distance apart from one another in a longitudinal direction along the inner catheter.

19. The method of claim 15, wherein each of the plurality of electrode disks are variably spaced apart from one another in a longitudinal direction along the inner catheter.

20. The method of claim 15, wherein the method step of inserting the catheter further comprises inserting the catheter over a guidewire and into the target area.
